(19) United States
(12) Patent Application Publication
(10) Pub. No.: US 2019/0240143 A1
(43) Pub. Date: Aug. 8, 2019

(54) Sustained Release Pharmaceutical Dosage Form of Entecavir

(71) Applicant: AUCTA Pharmaceuticals, North Brunswick, NJ (US)

(72) Inventors: Enxian Lu, East Brunswick, NJ (US); Shoufeng Li, Basking Ridge, NJ (US); Shaoqiong Lyu, Shanghai (CN)

(73) Assignee: AUCTA Pharmaceuticals, North Brunswick, NJ (US)

(21) Appl. No.: 16/382,921

(22) Filed: Apr. 12, 2019

Related U.S. Application Data
(63) Continuation-in-part of application No. PCT/US17/56318, filed on Oct. 12, 2017.
(60) Provisional application No. 62/407,262, filed on Oct. 12, 2016.

(30) Foreign Application Priority Data
May 12, 2017 (CN) 201710333110.3

(51) Int. Cl. A61K 9/00; A61K 31/708; A61K 47/38; A61K 47/26

(52) U.S. Cl. CPC A61K 9/0004; A61K 9/0053; A61K 47/26; A61K 47/38; A61K 31/708

(57) ABSTRACT

This document discloses a sustained release dosage form for oral administration of entecavir in a subject. Also disclosed is a method of treating hepatitis virus B infection.

### FIELD OF THE INVENTION

This patent document discloses a sustained release dosage form of entecavir or its pharmaceutically acceptable salt. The sustained release dosage form allows for administration at an interval longer than for example 3 days and thus provides better patient compliance than conventional immediate release dosage form.

### BACKGROUND

Hepatitis B is a viral infection that attacks the liver and can cause both acute and chronic disease. Chronic hepatitis B infection can be treated with drugs, including oral antiviral agents. Treatment can slow the progression of cirrhosis, reduce incidence of liver cancer and improve long term survival. WHO recommends the use of oral treatments—tenofovir or entecavir, because these are the most potent drugs to suppress hepatitis B virus. They rarely lead to drug resistance as compared with other drugs, and have few side effects so require only limited monitoring. In most people, however, the treatment does not cure hepatitis B infection, but only suppresses the replication of the virus. Therefore, most people who start hepatitis B treatment must continue it for life.

Entecavir is an oral antiviral drug used in the treatment of hepatitis B virus (HBV) infection. Entecavir is a reverse transcriptase inhibitor, which prevents the hepatitis B virus from multiplying and reduces the amount of virus in the body. Currently, entecavir is commercially available as immediate release tablet (0.5 mg, 1 mg) and is administered once daily.

Oral controlled release system is typically adopted for once daily administration. Oral long-acting controlled release system for the administration of entecavir is not available so far. Controlled release system for long-acting use is typically injection, such as BYDUREON® (exenatide extended-release) for injectable suspension. However, delivery of the drug by injection is often associated with inconveniences and often leads to patient non-compliance.

Since most patients need a life time treatment, a need exists for alternative formulations for improved patience compliance. It is further desirable that the improved dosage form can be manufactured cost-effectively.

### SUMMARY

The dosage form described herein allows for reduced frequency of administration but still provides therapeutically effective blood levels of medication over an extended period of time.

An aspect of the patent document provides a dosage form for oral administration in a subject. The dosage form, administered once every treatment cycle of 7 days, includes entecavir in an amount of from about 0.2 mg to about 25 mg and a sustained release component.

In some embodiments, the dosage form provides a Cmax ranging from around 1.0 to about 20 ng/mL during the seven-day period. In some embodiments, the dosage form provides an in vitro release of less than about 30% of the entecavir within about 1 hour, wherein the in vitro release of the entecavir is measured according to USP dissolution apparatus 1, in 900 ml of PBS solution at pH 6.8 at 100 rpm. In some embodiments, the dosage form provides an in vitro release of: (a) from about 10% to about 50% of the entecavir within about 2 hours; and (b) more than about 60% the entecavir within about 12 hours.

In some embodiments, the dosage form contains the entecavir ranging from about 0.5 mg to about 15 mg in the dosage form. the plasma concentration of the entecavir after administration of the dosage form ranges from about 0.01 ng/ml to about 20 ng/ml.

In some embodiments, the sustained release component comprises a hydrophilic polymer selected from the group consisting of hydroxypropyl methyl cellulose (HPMC), polyethylene oxide (PEO), hydroxypropyl cellulose (HPC), polyvinylpyrrolidone (PVP), polyvinyl alcohol (PVA), xanthan gum, carrageenan, and any combination thereof, wherein the hydrophilic polymer is in an amount of about 10% to about 80% by weight in the dosage form.

In some embodiments, the sustained release component comprises hydroxypropyl methyl cellulose (HPMC) or a derivative thereof, wherein the ratio by weight between the entecavir and the HPMC ranges from about 1:4 to about 1:100.

In some embodiments, the dosage form further contains lactose, wherein the ratio by weight between the entecavir and the lactose ranges from about 1:10 to about 1:25. In some embodiments, the dosage form further contains a lubricant selected from the group consisting of magnesium stearate, stearic acid, sodium stearyl fumarate, and sodium lauryl sulfate, glyceryl palmitostearate, and any combination thereof. In some embodiments, the dosage form further contains a glidant selected from the group consisting of silicon dioxide, starch, talc and any combination thereof.

In some embodiments, the dosage form is an osmotic controlled release oral delivery system (OROS) comprising a core enclosing the entecavir or the pharmaceutically acceptable salt thereof, and a semi-permeable membrane coating. In some embodiments, the core further comprises an osmotic agent, a swelling agent, further wherein the semi-permeable membrane coating comprises a water-insoluble polymer and a water soluble polymer.

In some embodiments, the dosage form is a hydrophobic matrix dosage form (e.g. tablet), a controlled release granule or a controlled release coated pellet. In some embodiments, the dosage form is the hydrophobic matrix dosage form comprising a hydrophobic polymer. In some embodiments, the hydrophobic polymer is selected from the group consisting of ethylcellulose (EC), methylethyl cellulose (MEC), methylcellulose (MC), and wax.

In some embodiments, the dosage form is the controlled release coated pellet comprising an immediate-release pellet coated with an agent selected from the group consisting of ethyl cellulose, acrylic resin, ethyl acrylate-meth acrylic acid copolymer, ethyl acrylate-methyl methacrylate-methacrylic acid copolymers, ethyl acrylate-methyl methacrylate and copolymers.

Another aspect provides a method of treating a subject infected with hepatitis B virus infection or co-infected with hepatitis B and another viral or non-viral disease. The method includes administering a dosage form described herein. In some embodiments, the entecavir ranges from about 0.5 mg to about 15 mg.

Another aspect provides a method of manufacturing the pharmaceutical dosage form described herein, comprising: (a) mixing entecavir with lactose to obtain a first mixture, wherein the lactose has the big surface area; (b) mixing the first mixture with a second mixture to obtain a third mixture, wherein the second mixture comprises a glidant; and (c) mixing the third mixture with a lubricant. In some embodiments, the lactose is amorphous lactose monohydrate.

### BRIEF DESCRIPTION OF THE DRAWINGS

FIG. 1 shows the dissolution curves of the entecavir controlled release hydrophilic and hydrophobic systems.

FIG. 2 shows the dissolution curves of the entecavir OROS systems at pH 6.8.

FIG. 3 shows the dissolution profiles of entecavir controlled release granules at pH 6.8 medium.

FIG. 4 shows PK model establishment for entecavir.

FIG. 5 shows predicted PK profiles of 1.5 mg entecavir formulations with different release profiles. “12 hr” means formulation release 100% drug at zero-order release rate during 12 hr. IR means immediate release.

### DETAILED DESCRIPTION

This document discloses a sustained release dosage form of entecavir or its pharmaceutically acceptable salt. Advantages of the dosage form include better compliance and low cost manufacturing in comparison with conventional immediate release dosage forms.

While the following text may reference or exemplify specific embodiments of a dosage form or a method of manufacturing the dosage from, it is not intended to limit the scope of the dosage form to such particular reference or examples. Various modifications may be made by those skilled in the art, in view of practical and economic considerations, such as the amount of individual excipients and the manufacturing condition.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “about” as used herein refers to the referenced numeric indication plus or minus 10% of that referenced numeric indication.

The term “active ingredient” or “active pharmaceutical ingredient” (API) refers to a compound (e.g., entecavir) that can be used for treating a disorder or condition in a subject (e.g., a patient), or for preventing one or more symptoms of such disorder or condition in the subject. As used herein, entecavir refers to the compound of 2-amino-9-[(1S,3R,4S)-4-hydroxy-3-(hydroxymethyl)-2-methylidenecyclopentyl]-1H-purin-6-one. In some embodiments, entecavir includes the hydrate, complex, polymorph, solvate or enantiomer of the above compound.

The term “content uniformity” refers to the homogeneity of the entecavir content among dosage forms, e.g. tablets, after formulation. Content uniformity can be expressed as a relative standard deviation of content uniformity of, for example, less than about 15%, less than about 10%, or less than about 5%.

The term “patient compliance” refers to the degree to which patient correctly follows medical advice.

The term “excipient” refers to any inert substance that may have specific functions to the drug (e.g., swelling agent, controlling-release, osmotic agent).

The term “direct compression” refers to a process where tablets are compressed directly from the mixture of the active ingredient and excipients without any preliminary treatment. The mixture to be compressed generally has adequate flow properties and coherence under pressure thus making pretreatment unnecessary.

The term “wet granulation” refers to a process of using a liquid binder to lightly agglomerate the powder mixture.

The term “release”, “released”, “releasing”, and the like, when used in connection with a pharmaceutical compression or dosage form, refers to the process or the portion of the active ingredient that leaves the dosage form following contact with an aqueous environment. Unless otherwise indicated, the quantity of an active ingredient released from a dosage form is measured by dissolution testing in water as described in this invention. The results of the dissolution testing are reported as % (w/w) released as a function of time or as the release time. In some embodiments, complete release of an active ingredient occurs when at least 90% of the active ingredient has been released from the dosage form.

The term “immediate-release” refers to those which disintegrate rapidly and/or get dissolved to release the medicaments or active ingredient.

The term “controlled release” or “sustained-release” refers to a pharmaceutical formulation which releases an active ingredient over an extended period of time and provides a relatively constant plasma concentration of the active ingredient.

The term “Cmax” or “peak plasma exposure”, expressed in ng/mL, refers to the point of maximum concentration of drug in plasma.

The term “area under curve (AUC)” or “total plasma exposure”, expressed in μg·hr/mL, refers to the total integrated area under plasma level time profile and expresses the total amount of the active ingredient that comes into systemic circulation after administration.

The term “subject” refers to a mammal, such as an animal or a human. Hence, the methods disclosed herein can be useful in human therapy and veterinary applications. In one embodiment, the subject is an animal. In another embodiment, the subject is a human.

The term “sustained release” refers to the result or effect that the therapeutically active medicament or active ingredient is released from the composition at a controlled rate such that therapeutically effective blood or plasma levels of the medicament or active ingredient are maintained over an extended period of time, e.g. providing a 1, 2, 3, 4, 5, 6 or 7-day therapeutic effect. A “sustained release component” is an agent or a combination of agents that enable the “sustained release” of the active ingredient.

The term “treat” or “treating” refers to attain or attaining a beneficial or desired result, such as a clinical result. In some embodiments, the beneficial or desired result is any one or more of the following: inhibiting or suppressing the onset or development of a condition, reducing the severity of the condition, reducing the number or severity of symptoms associated with the condition, increasing the quality of life of a patient suffering from the condition, decreasing the dose of another medication required to treat the condition, enhancing the effect of another medication a patient is taking for the condition, and prolonging the survival of a patient having the condition.

The term “therapeutically effective” means that a blood or plasma concentration or an amount of an active ingredient is effective to prevent, alleviate or ameliorate symptoms of disease or prolong the survival of the subject being treated.

An aspect of the document provides a pharmaceutical dosage form of entecavir for oral administration in a subject. The dosage form includes entecavir in an amount from about 0.2 mg to about 25 mg and a sustained release component. The dosage form is administrated at an interval longer than 3 days. In some embodiments, the dosage form is administered once every 2, 3, 4, 5, 6 or 7 days.

The dosage form can be of any suitable type. No-limiting examples include tablet, granule, bead, gel-caps, and pellet.

The amount of entecavir in the dosage form can vary depending on the administration schedule and the specific disease condition of a subject. Exemplary embodiments of the amount of entecavir in the dosage form include from about 0.2 to about 20 mg, from about 0.2 to about 15 mg, from about 0.4 to about 25 mg, from about 0.4 to about 20 mg, from about 0.5 to about 25 mg, from about 0.5 mg to about 15 mg, from about 0.5 to about 10 mg, from about 0.5 to about 5 mg, from about 0.8 to about 25 mg, from about 0.8 to about 15 mg, from about 1.0 to about 25 mg, and from about 1.0 to about 15 mg. In some embodiments, the dosage form contains about 0.4, about 0.5, about 0.6, 0.8, about 1.0, about 1.2, about 1.5, about 2.0, about 2.5, about 3.0, about 4.0, about 5.0, about 8.0, about 10.0, about 12.0, about 15.0 mg, about 20, about 25 of entecavir.

The dosage form provides a desirable sustained release of entecavir. The release can be measure by known methods such as USP Apparatus 1 or 2. In some embodiments, the in vitro release testing is conducted according to USP dissolution apparatus 1, in 900 ml of pH 6.8 at 100 rpm. Under such a USP dissolution apparatus 1 testing conditions, the release of entecavir within about 1 hour, within about 2 hours, or within about 12 hours from the dosage form described in this patent document after contacting an aqueous medium includes the following: in some embodiments, less than about 40%, less than about 35%, less than about 30%, less than about 25%, less than about 20% less than about 15%, or less than about 10% of the entecavir is released from the sustained release dosage form within about 1 hour; in some embodiments, from about 10% to about 70%, from about 10% to about 60%, from about 10% to about 50%, from about 10% to about 40%, from about 10% to about 30%, from about 20% to about 60%, from about 20% to about 50%, from about 20% to about 40%, from about 30% to about 60%, from about 30% to about 50%, or from about 30% to about 40% of the entecavir is released from the sustained release dosage form within about 2 hours; in some embodiments, from about 30% to about 70%, from about 30% to about 80%, from about 30% to about 90%, from about 30% to about 99%, from about 40% to about 70% from about 40% to about 80%, from about 40% to about 90%, from about 40% to about 99%, from about 50% to about 70% from about 50% to about 80%, from about 50% to about 90%, or from about 40% to about 99% of the entecavir is released from the sustained release dosage form within about 12 hours; and/or in some embodiments, from about 30% to about 70%, from about 30% to about 80%, from about 30% to about 90%, from about 40% to about 70%, from about 40% to about 80%, from about 40% to about 90%, from about 50% to about 70%, from about 50% to about 80%, from about 50% to about 90%, from about 60% to about 70%, from about 60% to about 80%, from about 60% to about 90%, from about 70% to about 80%, from about 70% to about 90%, from about 75% to about 80%, from about 75% to about 85%, or from about 80% to about 85%, or from about 85% to about 90% of the entecavir is released from the sustained release dosage form between about 7, 8, or 9 hours and about 12 hours. In an exemplary embodiment, about 85% of the entecavir is released between about 9 hours and about 12 hours, which also implies that less than about 85% of the entecavir is released within about 9 hours after the dosage form contacts an aqueous medium.

In some embodiments, less than about 10%, less than about 20%, less than about 30%, or less than about 40% of the entecavir was released, in vitro, from the dosage form within about 1 hour. In some embodiments, the dosage form provides an in vitro release of from about 10% to about 50% (but less than about 55% or 60%) of the entecavir within about 2 hours and, after such time, more than about 50%, more than about 60%, or more than about 70% in vitro release of entecavir is achieved from the dosage form within about 12 hours. The in vitro release of the entecavir is measured according to USP dissolution apparatus 1, in 900 ml of PBS solution at pH 6.8 at 100 rpm.

In some embodiments, the in vitro release testing is conducted according to USP dissolution apparatus 1, in 900 ml of pH 6.8 at 100 rpm. In some embodiments, less than about 70%, less than about 60%, less than about 50%, or less than about 45% of the entecavir is released, in vitro, from the dosage form within about 1 hour. In some embodiments, from about 20 to about 70%, from about 30 to about 60%, from about 30 to about 55%, or from about 30 to about 50% of the entecavir is released, in vitro, from the dosage form within about 1 hour. In some embodiments, from about 10% to about 90%, from about 10% to about 85%, from about 10% to about 80%, or from about 10% to about 75% of the entecavir is released from the sustained release dosage form within about 2 hours.

Typically, a weekly dosing for an oral form is not feasible due to GI transit times and physiology. Monthly or even longer dosing regimen is possible with controlled release depot system via injectable forms of microsphere. However, some injectable formulations are typically associated with a higher Cmax due to the much higher drug loading and burst release.

The dosage form described herein overcomes the problem. The sustained release of entecavir is a factor impacting the Cmax. Without being limited to any particular theory, it is also postulated that the deposit of entecavir into cells also reduce its blood or plasma concentration and Cmax. The cells serve a secondary release mechanism by releasing entecavir over time to provide extended therapeutic effect.

The dosage form provides a Cmax ranging from about 1.0 to about 20 ng/mL when the dosage form is administered once every 3, 4, 5, 6, or 7 days. In some embodiments, the Cmax may vary depending on the dosage and the interval between administrations of the entecavir dosage form. In some embodiments, the Cmax ranges from about 0.05 to about 20 ng/mL, from about 1.0 to about 15 ng/mL, from about 2.0 to about 15 ng/mL, from about 2.0 to about 10 ng/mL, or from about 2.0 to about 5.0 ng/mL.

In some embodiments, the dosage form is administered once every seven days and the Cmax is less than about 20 ng/mL, less than about 15 ng/mL, less than about 10 ng/mL, less than about 5.0 ng/mL, less than about 4.5 ng/mL, or less than about 4.0 ng/mL. Meanwhile, the plasma concentration of entecavir remains at therapeutically effective levels.

In some embodiments, the Cmin at end of a period or treatment cycle (e.g. 1 week) ranges from about 0.01 to about 0.8 ng/ml, from about 0.05 to about 0.8 ng/ml, from about 0.1 to about 0.8 ng/ml, from about 0.2 to about 0.8 ng/ml, from about 0.3 to about 0.8 ng/ml, from about 0.4 to about 0.8 ng/ml, from about 0.5 to about 0.8 ng/ml, or from about 0.6 to about 0.8 ng/ml.

The plasma concentration of entecavir of the dosage form maintains at therapeutically effective level with tolerable side effect. In some embodiments, during the interval after the administration of the sustained release dosage form described in this patent document, the plasma concentration of the entecavir ranges from about 0.01 ng/ml to about 20 ng/ml, from about 0.02 to about 15 ng/ml, from about 0.05 to about 15 ng/ml, from 0.05 ng/ml to about 10 ng/ml, from about 0.05 ng/ml to about 5 ng/ml, about 0.1 ng/ml to about 20 ng/ml, from about 0.1 to about 15 ng/ml, from about 0.1 to about 10 ng/ml, about 0.1 ng/ml to about 5 ng/ml, from about 0.1 to about 3 ng/ml, about 0.3 ng/ml to about 20 ng/ml, from about 0.3 to about 15 ng/ml, from about 0.3 to about 10 ng/ml, about 0.3 ng/ml to about 5 ng/ml, from about 0.3 to about 3 ng/ml, about 0.1 ng/ml to about 20 ng/ml, from about 0.5 to about 15 ng/ml, from about 0.5 to about 10 ng/ml, about 0.5 ng/ml to about 5 ng/ml, from about 0.5 to about 3 ng/ml, about 1 ng/ml to about 20 ng/ml, from about 1 to about 15 ng/ml, from about 1 to about 10 ng/ml, about 1 ng/ml to about 5 ng/ml, or from about 1 to about 3 ng/ml. In some embodiments, the interval is 1, 2, 3, 4, 5, 6, or 7 days. In some embodiments, the dosage form of entecavir is administered once every 7 days.

The dosage form has a desirable content uniformity. In some embodiments, the content uniformity has a relative standard deviation of less than about 20%, less than about 15%, less than about 10%, less than about 5%, less than about 2%, or less than about 1%.

The sustained release dosage can be in the form of, for example, a dosage form containing a hydrophilic sustained release agent, or a dosage form containing a hydrophobic sustained release agent. Additional exemplary dosage forms include Osmotic Controlled Release Oral Delivery System (OROS), controlled release granules and controlled release coated pellet. The formulation of the dosage form described herein also applies to salt, hydrate, complex, polymorph, solvate and enantiomer of entecavir. In some exemplary embodiments, the sustained release component includes acetate cellulose, HPMC, EC and Surelease® etc. Exemplary processes for preparing the sustained dosage form include direct compression, dry granulation, wet granulation, melt extrusion, extrusion/spheronization and coating.

In some embodiments, the sustained release component includes a hydrophilic polymer. Non-limiting examples of the hydrophilic polymer include hydroxypropyl methyl cellulose (HPMC), polyethylene oxide (PEO), hydroxypropyl cellulose (HPC), polyvinyl pyrrolidone (PVP), polyvinyl alcohol (PVA), xanthan, gum, carrageenan. In some embodiments, the hydrophilic polymer is HPMC or a derivative thereof. In some embodiments, the hydrophilic polymer is HPMC K15M (METHOCEL™ K15M Premium CR Hydroxypropyl Methylcellulose). In some embodiments, the hydrophilic polymer is HPMC K100M (METHOCEL™ K100M Premium CR Hydroxypropyl Methylcellulose) with viscosity range (75,000 mPa·s-140,000 mPa·s). In some embodiments, the hydrophilic polymer is K4M (METHOCEL™ K4M Premium CR Hydroxypropyl Methylcellulose) with viscosity range (2,500 mPa·s-5,000 mPa·s). In some embodiments, the HPMC has a particle size ranging from about 100 to about 300 micrometers, from about 150 to about 250 micrometers, or from about 170 to about 250 micrometers. In some embodiments, the HPMC has a viscosity ranging from about 1000 to about 150,000 cps, from about 2500 to about 150,000 cps, from about 3000 to about 150,000 cps, from about 10,000 to about 25,000 cps, from about 13,000 to about 30,000 cps, from about 10,000 to about 150,000 cps, from about 50,000 to about 150,000 cps, from about 75,000 to about 150,000 cps, or from about 75,000 to about 140,000 cps.

The amount of the hydrophilic polymer may vary depending on the dosage of entecavir and the amount of other excipients. In some embodiments, the hydrophilic polymer is present in an amount from about 10% to about 90%, from about 10% to about 80%, from about 20% to about 80%, from about 20% to about 70%, from about 20% to about 60%, from about 20% to about 50%, from about 30% to about 70%, from about 30% to about 60%, or from about 30% to about 50% by weight of the dosage form. In some embodiments, the ratio by weight between entecavir and the hydrophilic polymer ranges from about 1:20 to about 1:85, from about 1:30 to about 1:75, or from about 1:40 to about 1:65.

The dosage form described herein can further include a filler such as lactose and mannitol which facilitate the even distribution of entecavir in the dosage form. In some embodiments, the acceptance value for the content uniformity of entecavir dosage form is less than about 20, less than about 18 or less than about 15. In some embodiments, the dosage form has a relative standard deviation of content uniformity of less than about 5%, less than about 4%, less than about 3%, less than about 2%, or less than about 1%. In some embodiments, the filler is lactose. In some embodiments, the filler is amorphous lactose monohydrate, which can be prepared for example by spray-drying process. In some embodiments, the lactose is substantially free from water or contains less than about 1%, less than about 0.5%, less than about 0.3%, or less than about 0.1% of water by weight. Preferably, the lactose is in a form that provides a high surface area for contacting and/or absorbing entecavir. For example, the lactose can be processed through spray-dry procedure to achieve a powdery state with a high surface area but a low amount of water.

In some embodiments, the sustained release component comprises hydroxypropyl methyl cellulose (HPMC) or a derivative thereof. In some embodiments, the ratio by weight between the entecavir and the HPMC ranges from about 1:1 to about 1:100, from about 1:1 to about 1:75, from about 1:1 to about 1:50, from about 1:1 to about 1:25, from about 1:1 to about 1:20, from about 1:1 to about 1:15, from about 1:1 to about 1:10, from about 1:1 to about 1:8, from about 1:1 to about 1:6, from about 1:1 to about 1:5, from about 1:1 to about 1:4, from about 1:1 to about 1:3, or from about 1:1 to about 1:2, all subranges included. In some further exemplary embodiments, the ratio by weight between the entecavir and the HPMC is about 1:1, about 1:2, about 1:3, about 1:4, about 1:5, about 1:8, about 1:10, about 1:15, about 1:20, about 1:25, about 1:30, about 1:40, or about 1:50.

In some embodiments, the ratio be weight between entecavir and lactose ranges from about 1:10 to about 1:25, from about 1:11 to about 1:20, from about 1:15 to about 1:20, from about 1:10 to about 1:15, or from about 1:12 to about 1:15.

The dosage form can also include a lubricant. Non-limiting examples of lubricant include magnesium stearate, stearic acid, sodium stearyl fumarate, sodium lauryl sulfate, glyceryl palmitostearate, and any combination thereof.

The dosage form can also include a glidant. Non-limiting examples of glidant include silicon dioxide, starch, talc and any combination thereof.

The dosage form can contain be a pH modifier or buffering agent. In some embodiments, the buffering agent is coated on the dosage form. Non-limiting examples of the buffering agent include sodium citrate, citric acid, fumaric acid, tartaric acid, potassium citrate, sodium bicarbonate, potassium bicarbonate, sodium dihydrogen phosphate, disodium hydrogen phosphate, and sodium hydroxide and potassium dihydrogen phosphate.

Non-limiting examples of the dosage form also include hydrophobic matrix tablet, controlled release granule and controlled release coated pellet.

In some embodiments, the dosage form is an osmotic controlled release oral delivery system (OROS) comprising a semi-permeable membrane coating and a core comprising the entecavir, or the pharmaceutically acceptable salt or hydrate thereof. In some embodiments, the core contains one or more agents selected from an osmotic agent (such as sorbitol, mannitol, lactose, salt etc.), a swelling agent (such as polyethylene oxide, hydroxyethyl cellulose, croscarmellose sodium, polyvinyl polypyrrolidone etc.). In some embodiments, the semi-permeable membrane coating comprises substantially a water-insoluble polymer (e.g. Cellulose Acetate) and a water soluble polymer (e.g. Klucel EF).

In some embodiments, the dosage form is a hydrophobic matrix tablet containing hydrophobic polymer selected from the group consisting of ethylcellulose (EC), methylethyl cellulose (MEC), methylcellulose (MC), and wax. The hydrophobic polymer is present in about 10% to about 80% of the tablet by weight.

In some embodiments, the dosage form is a controlled release coated pellet containing an immediate-release pellet coated with an agent selected from ethyl cellulose, acrylic resin, ethyl acrylate-meth acrylic acid copolymer, ethyl acrylate-methyl methacrylate-methacrylic acid copolymers, ethyl acrylate-methyl methacrylate copolymers, and any other suitable polymers.

The dosage form can be manufactured by various processes depending on the specific form and the subject to be treated. For example, a dosage form such as a tablet can be prepared by direct compression, dry granulation, wet granulation and melt granulation. A dosage form can also be prepared via membrane coating or fluid bed coating (e.g. controlled release coated pellet or osmotic controlled release oral delivery system). A dosage form such as controlled release granule or beads can be prepared by extrusion-spheronization or melt extrusion technology.

In some embodiments, a dosage form can be manufacture via the following method:

(a) mixing entecavir with a filler to obtain a first mixture, wherein the filler allows for even distribution of entecavir in the dosage form;

(b) mixing the first mixture with a second mixture to obtain a third mixture, wherein the second mixture comprises a glidant; and

(c) mixing the third mixture with a lubricant.

In some embodiments of the above method, the filler is lactose. Preferably, the lactose contains minimum amount of water as stated above. In some embodiments, the lactose is spray dried to remove the moisture and maximize its surface area, such as Flowlac. In some embodiments, the first mixture is filtered through a 30-50 mesh screen. Other components in the second mixture and/or third mixture can also be filtered to remove large particles. For example, the hydrophilic polymer such as HPMC or its derivative and the glidant can be filtered through a 20-40 mesh screen. In some embodiments, the method further include coating the solid form of step (c) with a pH modifier or buffering agent which are described above. in some embodiments, the buffering agent contains citric acid.

In some exemplary embodiments, the method of preparation is as follows:

(a) Passing the entecavir powder and lactose through 30-50 mesh to obtain a first mixture, wherein the lactose is amorphous lactose monohydrate (e.g. by spray drying);

(b) mixing the first mixture with a second mixture to obtain a third mixture, wherein the second mixture comprises a glidant, hydrophilic polymer and a filler; and

(c) mixing the third mixture with a lubricant and forming a solid form (e.g. a tablet).

Another aspect of this patent document provides a method of treating a subject infected with hepatitis B virus infection or co-infected with hepatitis B and another viral or non-viral disease comprising administering a dosage form of any of the above described embodiments. In some embodiments, the entecavir ranges from about 0.2 mg to about 25 mg or from about 0.5 mg to about 15 mg. In some embodiments, the dosage form is administered once every 7 days. In some embodiments, the subject is a human.

This method is also directed to the treatment of hepatitis B virus infection in combination with one or more other pharmaceutically active agents. Suitable pharmaceutically active agents for this purpose include one or more antiviral agents, for example, didanosine, lamivudine, abacavir, adefovir, adefovir dipivoxil, famciclovir, (2R,4R)-4-(2,6-diamino-9H-purin-9-yl)-2-hydroxymethyl-1,3-dioxolane (DAPD), hepatitis B immunomodulating proteins (EHT 899 from Enzo Biochem), emtricitabine, 1-(2-deoxy-2-fluoro-β-D-arabinofuranosyl) thymine (FMAU), GLQ-223 (Compound A, alpha-trichosanthin), epavudine (L-dT), epcitabine (L-dC), ribavirin, tenofovir (PMPA), 2′,3′-dideoxy-2′,3′-didehydro-beta-L(−)-5-fluorocytidine [L (−)Fd4C], as well as other fluoro L- and D-nucleosides. Suitable pharmaceutically active agents for this purpose also include one or more immunomodulators, for example, alpha interferon, beta interferon, pegylated interferon, thymosin alpha, and hepatitis B vaccines such as HBV/MF59, Hepagene and Theradigm-HBV. The dosage form described herein can be co-administered or sequentially administrated with a secondary antiviral agent or agents according to a prescribed schedule.

### EXAMPLES

### Example 1

Entecavir and excipients shown in table 1 were mixed. Then the tablets were prepared by direct compression with 7 mm (diameter) round tooling on a single-punch tablet press.

TABLE 1
Formulation of entecavir hydrophilic matrix tablet in Example 1

| Ingredient (mg) | 20160926-1 | 20160926-2 |
|---|---|---|
| Entecavir monohydrate | 3.7 | 3.7 |
| Lactose monohydrate | 61.3 | 61.3 |
| AvicelPH102 | 72 | 72 |
| HPMC K15M | 60 | — |
| HPMC K100M | — | 60 |
| Magnesium stearate | 3 | 3 |
| Total | 200 | 200 |

### Example 2

Entecavir and excipients shown in table 2 were mixed. Then the tablets were prepared by direct compression with 7 mm (diameter) round tooling on a single-punch tablet press.

TABLE 2
Formulations of entecavir hydrophobic matrix tablet in Example 2

| Ingredient (mg) | 20160926-3 | 20160926-4 | 20160926-5 |
|---|---|---|---|
| Entecavir monohydrate | 3.7 | 3.7 | 3.7 |
| CaH2PO4 | 113.3 | 113.3 | 83.3 |
| EC 10FP | 60 | 90 | 90 |
| EC 20P | | | 60 |
| HPMC K100 | 20 | 20 | 20 |
| Magnesium stearate | 3 | 3 | 3 |
| Total | 200 | 200 | 200 |

Dissolution test: The dissolution of the formulation was carried out by using USP Dissolution Apparatus I (basket). The dissolution test employed a speed at 100 rpm and 900 ml of pH 6.8 PBS solution as the dissolution medium maintained at 37±0.5° C. The dissolution samples were analyzed by a High Performance Liquid Chromatography (HPLC) analytical method at the time of 1, 2, 4, 6, 9, 12, 14, 16, 18 and 24 hour. The dissolution curves of the five formulations in Example 1 and 2 are presented in FIG. 1.

### Example 3

The OROS tablet cores were prepared by direct compression with 5.28*10.56 mm oval tooling on a single-punch tablet press. The coating solution was prepared by dissolving cellulose acetate and Klucel in methanol/acetone, then tablet cores were coated with the 2% (w/w) solution. A single hole (0.55 mm) was drilled in the end of the band of the oval tablet.

TABLE 3
Formulation of entecavir OROS tablet in Example 3

| Formulation | Compositions (mg) | 20160928-1 |
|---|---|---|
| Tablet core | Entecavirmonohydrate | 3.7 |
| | Sorbitol | 166.5 |
| | Hydroxyethy cellulose MF | 16 |
| | Copovidone (VA64) | 12 |
| | Magnesium stearate | 2 |
| | Total | 200 |
| Film Coating | Cellulose Acetate | 1.8% |
| | Klucel EF | 0.2% |
| | Methanol | 20% |
| | Acetone | 78% |
| | Coating weight gain | 7%, 10% |
| Tablet Weight | — | 214/220 |

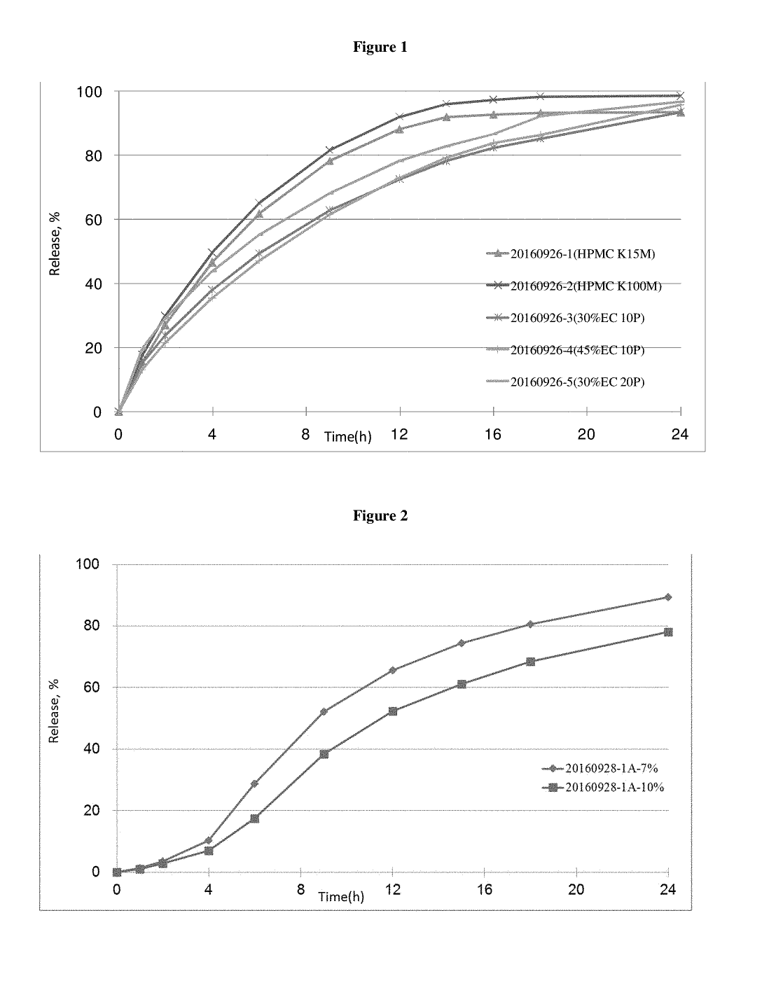

The dissolution curves of the OROS formulations in Example 3 are presented in FIG. 2.

### Example 4

The granules shown in Table 4 were prepared by melt extrusion approach. Entecavir and compritol 888 were physically mixed, and then melt extruded. The melt extrudant was milled and sieved to get the required particle size granule.

TABLE 4
Formulation of entecavir controlled release granule in Example 4

| Formulation | Compositions (mg) | 20161010-1 | 20161010-2 |
|---|---|---|---|
| Internal | Entecavirmonohydrate | 3.7 | 3.7 |
| | Compritol 888 | 20 | 40 |
| External | Lactose monohydrate | 126.3 | 106.3 |
| Tablet Weight | — | 150 | 150 |

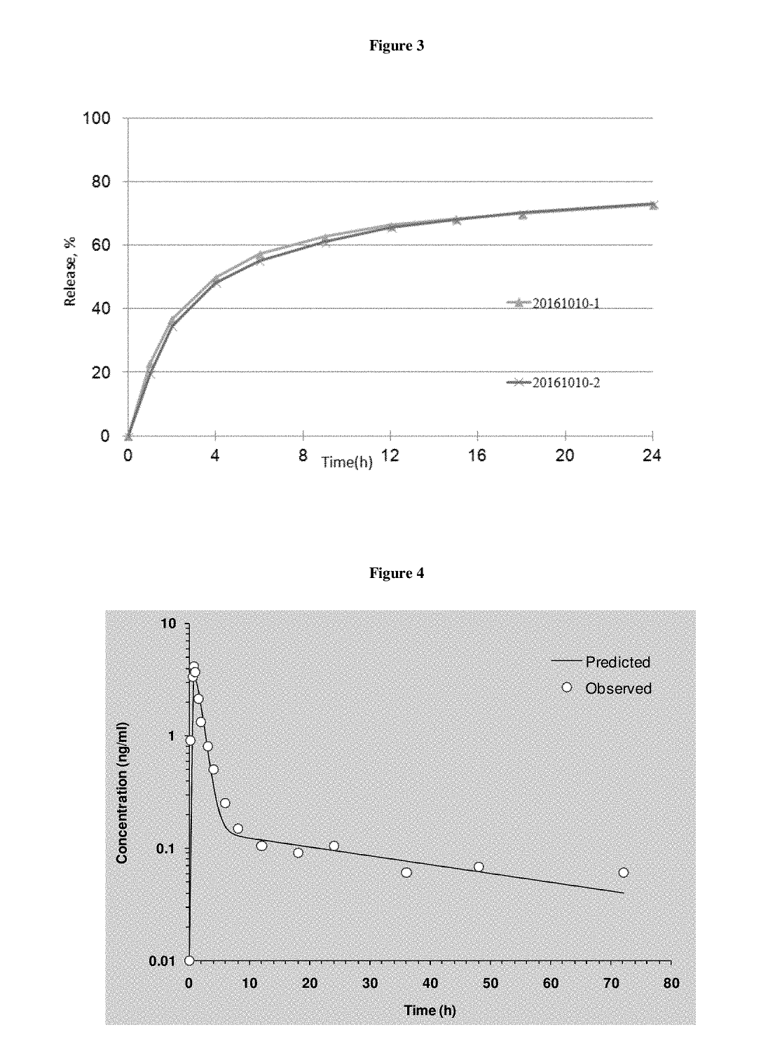

The dissolution curves of the granule formulations in Example 4 are presented in FIG. 3.

### Example 5

The coated pellet shown in Table 5 were prepared by fluid bed coating process. Drug coating solution contains drug, Hypromellose E3 and Talc. The sugar spheres were coated with drug solution in fluid bed. Then the spheres containing drug were coated with surlease to obtain the controlled release coated pellet.

TABLE 5
Formulation of entecavir controlled release coated pellet in Example 5

| Formulation | Compositions (mg) | 20161012-1/2/3 |
|---|---|---|
| Drug coating | Entecavirmonohydrate | 3.7 |
| | Sugar spheres | 120 |
| | Hypromellose E3 | 1 |
| | Talc | 3 |
| | Purified water | — |
| Functional coating | Surelease ® | 6%, 9%, 12%, 15% |

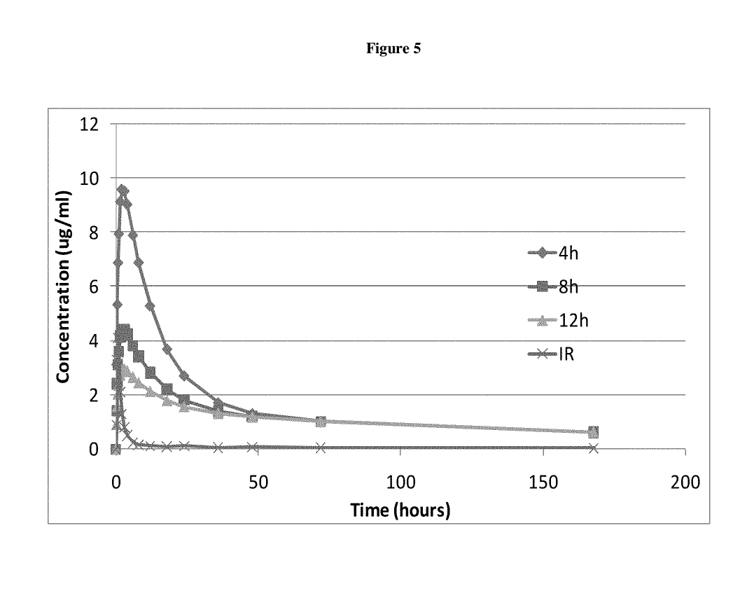

Pharmacokinetic (PK) simulation was used to design the desired release profiles. PK model for entecavir was established based on IR tablet PK data and the prediction curve fits well with observed curve as shown in FIG. 4. Then the 1.5 mg controlled release formulation with different release profiles were put into the pK model and the predicted PK profile was obtained and shown in FIG. 5. The 1.5 mg formulation with 8 h and 12 hr release has the Cmax of 4.4 ng/ml and 2.94 ng/ml, respectively, and Cmin at 1 week of 0.62 ng/ml and 0.63 ng/ml, respectively, which fits the requirement.

### Example 6

Flowlac (lactose monohydrate) was processed through spray-dry procedure to achieve a powdery state that provides a high surface area for contacting and/or absorbing entecavir. The processed lactose monohydrate has a high surface area. This topical characteristic can improve the uniformity in blending process. Table 6 shows the ratio by weight between entecavir and lactose ranging from about 1:10 to about 1:25 perform qualified blending uniformity (BU).

TABLE 6
The influence of different amounts of lactose on contect uniformity (BU)

| Procedure | Compositions | Formulation 1 | Formulation 2 | Formulation 3 | Formulation 4 | Formulation 5 | Formulation 6 |
|---|---|---|---|---|---|---|---|
| Step 1 | Entecavir | 1:5 | 1:10 | 1:15 | 1:20 | 1:25 | 1:28 |
| | monohydrate:spray- | sieving | sieving | sieving | sieving | sieving | sieving |
| | dry | | | | | | |
| | lactose | | | | | | |
| Step 2 | MCC + HPMC + | Mixing for 10 min | | | | | |
| | Silicon dioxide | | | | | | |
| Step 3 | Magnesium | Mixing for 5 min | | | | | |
| | stearate | | | | | | |
| BU | — | unqualified | qualified | qualified | qualified | qualified | unqualified |
| Evaluation | | | | | | | |

### Example 7

The invention further provides an entecavir medicinal composition, the ratio by weight between entecavir and the hydrophilic polymer ranges from about 1:30 to about 1:75, the ratio by weight between entecavir and the filler ranges from about 1:35 to about 1:90, the ratio by weight between entecavir and the lubricant ranges from about 1:0.5 to about 1:1.5, the ratio by weight between entecavir and the glidant ranges from about 1:0.5 to about 1:1.5. Table 7 shows the entecavir matrix tablet containing different type of hydrophilic polymer. The dissolution data of the six formulations are presented in Table 8.

TABLE 7
Formulation of entecavir hydrophilic matrix tablet in Example 7

| Compositions (mg) | 20161114-1 | 20161121-1 | 20161121-2 | 20161121-3 | 20161121-4 | 20160926-2 |
|---|---|---|---|---|---|---|
| Entecavir | 1.6 | 1.6 | 1.6 | 1.6 | 1.6 | 1.6 |
| Monohydrate | | | | | | |
| Lactose monohydrate | 20 | 20 | 20 | 20 | 20 | 20 |
| (Flowlac) | | | | | | |
| MCC | 96.4 | 96.4 | 96.4 | 96.4 | 96.4 | 96.4 |
| HPMC K15M | 80 | — | — | — | — | — |
| HPMC K4M | — | 80 | — | — | — | — |
| HPMC K100M | | | | | | 80 |
| PEO 301 | — | — | 80 | — | — | — |
| PEO N-60K | — | — | — | 80 | — | — |
| PEO 1105 | — | — | — | — | 80 | — |
| Magnesium stearate | 1.0 | 1.0 | 1.0 | 1.0 | 1.0 | 1.0 |
| Silicon dioxide | 1.0 | 1.0 | 1.0 | 1.0 | 1.0 | 1.0 |
| Total | 200 | 200 | 200 | 200 | 200 | 200 |

TABLE 8
The dissolution data of entecavir hydrophilic matrix tablet in Example 7 (% w/w)

| Release | | | | | | |
|---|---|---|---|---|---|---|
| (hour) | 20161114-1 | 20161121-1 | 20161121-2 | 20161121-3 | 20161121-4 | 20160926-2 |
| 1 | 17.8 | 19.6 | 18.4 | 21.8 | 24.9 | 17.7 |
| 2 | 29.9 | 31.6 | 32.7 | 38.3 | 44.7 | 29.7 |
| 4 | 49.0 | 49.7 | 56.7 | 66.4 | 76.2 | 49.5 |
| 6 | 63.6 | 63.5 | 74.8 | 85.4 | 91.8 | 65.0 |
| 9 | 79.6 | 78.8 | 90.5 | 98.4 | 100.1 | 81.5 |
| 12 | 90.4 | 89.1 | 95.1 | 100.6 | 100.4 | 91.9 |
| 15 | 97.3 | 94.7 | 95.8 | 100.9 | 100.6 | 96.8 |
| 18 | 101.5 | 97.4 | 96.6 | 101.8 | 101.5 | 98.8 |
| 24 | 103.8 | 98.4 | 97.0 | 102.5 | 102.5 | 99.3 |

According to the dissolution result, The HMPC K4M, K15M and K100M perform perfect drug release behavior, especially the HPMC K15M due to less than about 20% of the entecavir within about 1 hour and better flowability than HPMC K100M.

### Example 8

TABLE 9
The influence of different HPMC K15M content on drug release

| Compositions | | | | | |
|---|---|---|---|---|---|
| (mg) | 20161114-1 | 20161128-1 | 20161128-2 | 20161128-3 | 20161128-4 |
| Entecavir | 1.6 | 1.6 | 1.6 | 1.6 | 1.6 |
| Monohydrate | | | | | |
| Lactose | 20 | 20 | 20 | 20 | 20 |
| MCC | 96.4 | 136.4 | 116.4 | 76.4 | 56.4 |
| HPMC K15M | 80 | 40 | 60 | 100 | 120 |
| Magnesium | 1.0 | 1.0 | 1.0 | 1.0 | 1.0 |
| stearate | | | | | |
| Silicon dioxide | 1.0 | 1.0 | 1.0 | 1.0 | 1.0 |
| Total | 200 | 200 | 200 | 200 | 200 |

The formulations to evaluate the influence of different HPMC K15M content on drug release are shown in Table 9. Entecavir powder and lactose were passed through 30 mesh to obtain a first mixture, which was mixed with MCC, HPMC K15M and Silicon dioxide for 15 min to obtain a second mixture. The second mixture was mixed with magnesium stearate for 5 min. A tablet was obtained by pressing with 7 mm round tooling.

The in-vitro dissolution data are presented in Table 10. The result shows the HPMC K15M content ranging from about 30% to about 60% perform a similar release within 9 hours.

TABLE 10
The dissolution data of different HPMC K15M content
entecavir matrix tablet in Example 8 (% w/w)

| Release | 20161114- | 20161128- | 20161128- | 20161128- | 20161128- |
|---|---|---|---|---|---|
| (hour) | 1 | 1 | 2 | 3 | 4 |
| 1 | 17.8 | 41.6 | 24.1 | 19.1 | 18.0 |
| 2 | 29.9 | 58.0 | 37.4 | 31.1 | 29.4 |
| 4 | 49.0 | 76.5 | 56.6 | 48.7 | 46.7 |
| 6 | 63.6 | 86.9 | 70.6 | 62.0 | 60.0 |
| 9 | 79.6 | 94.2 | 84.5 | 76.7 | 75.2 |
| 12 | 90.4 | 99.3 | 92.9 | 86.6 | 85.7 |
| 15 | 97.3 | 100.2 | 98.0 | 93.8 | 93.4 |
| 18 | 101.5 | 101.0 | 100.0 | 97.9 | 97.8 |
| 24 | 103.8 | 100.0 | 99.9 | 99.9 | 100.0 |

### Example 9

TABLE 11
The influence of different HPMC K4M and K100M content on drug release

| Compositions (mg) | 20161201-1 | 20161201-2 | 20161201-3 | 20161205-1 | 20161205-2 | 20161205-3 |
|---|---|---|---|---|---|---|
| Entecavir | 1.6 | 1.6 | 1.6 | 1.6 | 1.6 | 1.6 |
| Monohydrate | | | | | | |
| Lactose | 20 | 20 | 20 | 20 | 20 | 20 |
| MCC | 136.4 | 116.4 | 56.4 | 136.4 | 116.4 | 56.4 |
| HPMC K4M | 40 | 60 | 120 | — | — | — |
| HPMC K100M | — | — | — | 40 | 60 | 120 |
| Magnesium | 1.0 | 1.0 | 1.0 | 1.0 | 1.0 | 1.0 |
| stearate | | | | | | |
| Silicon dioxide | 1.0 | 1.0 | 1.0 | 1.0 | 1.0 | 1.0 |
| Total | 200 | 200 | 200 | 200 | 200 | 200 |

The preparation process is same as the above example. The result shows the HPMC K4M content ranging from about 30% to about 60% and the HPMC K100M content ranging from about 20% to about 60% provide a similar release of entecavir within 9 hours.

### Example 10

TABLE 12
The influence of different entecavir content on release

| Compositions | 20161113- | 20161114- | 20170322- | 20170322- |
|---|---|---|---|---|
| (mg) | 1 | 1 | 1 | 2 |
| Entecavir | 1.06 | 1.6 | 3.2 | 4.8 |
| Monohydrate | | | | |
| Lactose | 20 | 20 | 20 | 20 |
| MCC | 96.94 | 96.4 | 94.8 | 93.2 |
| HPMC K15M | 80 | 80 | 80 | 80 |
| Magnesium | 1.0 | 1.0 | 1.0 | 1.0 |
| stearate | | | | |
| Silicon dioxide | 1.0 | 1.0 | 1.0 | 1.0 |
| Total | 200 | 200 | 200 | 200 |

The preparation process is same as the above example. The result shows the entecavir content have no effect on its release behavior.

While the invention has been disclosed in some detail by way of illustration and example for purposes of clarity of understanding, it is apparent to those in the art that various changes may be made and equivalents may be substituted without departing from the true spirit and scope of the invention. Therefore, the description and examples should not be construed as limiting the scope of the invention.

1. A dosage form for oral administration in a subject, comprising entecavir or a pharmaceutically acceptable salt thereof in an amount from about 0.2 mg to about 25 mg and an sustained release component, wherein the dosage form achieves therapeutically effective plasma levels over a seven-day period when administered on a once weekly basis.

2. The dosage form of claim 1, which provides a Cmax ranging from about 1.0 to about 20 ng/mL during the seven-day period.

3. The dosage form of claim 1, which provides an in vitro release of less than about 40% of the entecavir within about 1 hour, wherein the in vitro release of the entecavir is measured according to USP dissolution apparatus 1, in 900 ml of PBS solution at pH 6.8 at 100 rpm. Page 7, line 10

4. The dosage form of claim 1, which provides an in vitro release of:
(a) from about 10% to about 70% of the entecavir within about 2 hours; and
(b) more than about 60% the entecavir within about 12 hours,
wherein the in vitro release of the entecavir is measured according to USP dissolution apparatus 1, in 900 ml of PBS solution at pH 6.8 at 100 rpm. Page 7, line 13

5. The dosage form of claim 1, wherein the entecavir ranges from about 0.5 mg to about 15 mg in the dosage form.

6. The dosage form of claim 1, which provides a plasma concentration of the entecavir after administration ranging from about 0.01 ng/ml to about 20 ng/ml.

7. The dosage form of claim 1, wherein the sustained release component comprises a hydrophilic polymer selected from the group consisting of hydroxypropyl methyl cellulose (HPMC), polyethylene oxide (PEO), hydroxypropyl cellulose (HPC), polyvinylpyrrolidone (PVP), polyvinyl alcohol (PVA), xanthan gum, carrageenan, and any combination thereof, wherein the hydrophilic polymer is in an amount of about 10% to about 80% by weight in the dosage form.

8. The dosage form of claim 1, wherein the sustained release component comprises hydroxypropyl methyl cellulose (HPMC), wherein the ratio by weight between the entecavir and the HPMC ranges from about 1:4 to about 1:100.

9. The dosage form of claim 1, further comprising lactose, wherein the ratio by weight between the entecavir and the lactose ranges from about 1:10 to about 1:25.

10. The dosage form of claim 1, further comprising a lubricant selected from the group consisting of magnesium stearate, stearic acid, sodium stearyl fumarate, and sodium lauryl sulfate, glyceryl palmitostearate, and any combination thereof.

11. The dosage form of claim 1, further comprising a glidant selected from the group consisting of silicon dioxide, starch, talc and any combination thereof.

12. The dosage form of claim 1, which is an osmotic controlled release oral delivery system (OROS) comprising a core enclosing the entecavir or the pharmaceutically acceptable salt thereof, and a semi-permeable membrane coating.

13. The dosage form of claim 12, wherein the core further comprises an osmotic agent, a swelling agent, further wherein the semi-permeable membrane coating comprises a water-insoluble polymer and a water soluble polymer.

14. The dosage form of claim 1, which is a hydrophobic matrix dosage form, a controlled release granule or a controlled release coated pellet.

15. The dosage form of claim 14, wherein the dosage form is the hydrophobic matrix dosage form comprising a hydrophobic polymer.

16. The dosage form of claim 15, wherein the hydrophobic polymer is selected from the group consisting of ethylcellulose (EC), methylethyl cellulose (MEC), methylcellulose (MC), and wax.

17. The dosage form of claim 14, wherein the dosage form is the controlled release coated pellet comprising an immediate-release pellet coated with an agent selected from the group consisting of ethyl cellulose, acrylic resin, ethyl acrylate-meth acrylic acid copolymer, ethyl acrylate-methyl methacrylate-methacrylic acid copolymers, and ethyl acrylate-methyl methacrylate copolymers.

18. A method of manufacturing the pharmaceutical dosage form of claim 1, comprising:
(a) mixing entecavir with lactose to obtain a first mixture, wherein the lactose is amorphous;
(b) mixing the first mixture with a second mixture to obtain a third mixture, wherein the second mixture comprises a glidant; and
(c) mixing the third mixture with a lubricant.

19. A method of treating a subject infected with hepatitis B virus infection or co-infected with hepatitis B and another viral or non-viral disease comprising administering a dosage form of claim 1.

20. The method of claim 19, wherein the entecavir ranges from about 0.5 mg to about 15 mg.

21. The dosage form of claim 1, which provides an in vitro release of less than about 60% of the entecavir within about 1 hour, wherein the in vitro release of the entecavir is measured according to USP dissolution apparatus 1, in 900 ml of PBS solution at pH 6.8 at 100 rpm.

22. The dosage form of 1, which provides an in vitro release of:
(a) from about 10% to about 80% of the entecavir within about 2 hours; and
(b) more than about 60% the entecavir within about 12 hours,
wherein the in vitro release of the entecavir is measured according to USP dissolution apparatus 1, in 900 ml of PBS solution at pH 6.8 at 100 rpm.

23. The dosage form of 8, wherein the ratio by weight between the entecavir and the HPMC ranges from about 1:1 to about 1:20.
